(19) United States
(12) Patent Application Publication
(10) Pub. No.: US 2018/0228145 A1
(43) Pub. Date: Aug. 16, 2018

(54) ABSORBENT BINDER COMPOSITION

(71) Applicant: Kimberly-Clark Worldwide, Inc., Neenah, WI (US)

(72) Inventors: John Gavin MacDonald, Decatur, GA (US); Dave Allen Soerens, Neenah, WI (US); David John Tyrrell, Milwaukee, WI (US)

(21) Appl. No.: 15/751,379

(22) PCT Filed: Aug. 30, 2016
(86) PCT No.: PCT/US2016/049472

§ 371 Date: Feb. 8, 2018

Related U.S. Application Data
(60) Provisional application No. 62/211,954, filed on Aug. 31, 2015.

(51) Int. Cl. A01N 25/04; A01N 25/08; A01N 59/16; A61L 26/00; A01N 33/12; A01N 59/00

(52) U.S. Cl. CPC A01N 25/04; A01N 25/08; A01N 59/16; A61L 26/0066; A61L 26/0061; A61L 26/0004; A61L 2300/11; A01N 33/12; A01N 59/00; A61L 26/008; A61L 2300/208; A61L 2300/104; A61L 2400/04; A61L 26/0014

(57) ABSTRACT

The present disclosure is directed towards an absorbent binder composition that includes a hydrophilic polymer which has the capability of post-application, moisture-induced cross-linking. The absorbent binder composition can include 1) a superabsorbent polymer material which can include at least 15 mass percent monoethylenically unsaturated polymer, such as carboxylic acid, sulphonic acid, or phosphoric acid, or salts thereof, and an acrylate or methacrylate ester that contains an alkoxysilane functionality and 2) a polyvalent metal cation having a valence of at least two. Upon loss of water by evaporation, the alkoxysilane functionality forms a silanol functional group which condenses to form a crosslinked polymer. Upon exposure of the absorbent binder composition to a biological material, such as urine, blood, or feces, the biological material can interact with the polyvalent metal cation and can serve as a catalyst to accelerate the polymer cross-linking and gelation of the polymer.

### RELATED APPLICATIONS

The present application claims priority to U.S. Provisional Application No. 62/211,954, filed Aug. 31, 2015, the contents of which are hereby incorporated by reference in a manner consistent with the present application.

### BACKGROUND OF THE DISCLOSURE

The present disclosure relates to an absorbent binder composition which can absorb a contaminant such as a biological material. The biological material can be located on a variety of surfaces such as hard surfaces or on the skin of a human.

Hospitals require that all hard surfaces in patient and treatment rooms be cleaned and disinfected. Targeted soils include, but are not limited to, biological materials such as blood, urine and fecal matter. A two-step process is generally used to clean such material from hard surfaces, one step for cleaning and another for disinfecting. Cleaning the biological materials routinely involves disposable wet wipes and/or non-disposable cloths that must be sanitized by laundering. Biological materials can be anywhere in the room, making cleanup with a wet wipe or cloth rather challenging (e.g., walls, equipment, connecting cables, etc.). After cleaning the biological materials from all hard surfaces, the same surfaces must be disinfected to eliminate infectious microorganisms left behind by the biological materials. With an increase in multi-drug resistant organisms such as MRSA, there is a mandated time in which the disinfectant must make contact with infectious microorganisms.

Commonly, two staff members are needed to both clean and disinfect the room which is time consuming and costly. Furthermore, the amount of time needed to for the cleaning and/or disinfecting solution(s) to dry can become problematic if it results in a delay for when the patient and treatment rooms can be utilized again (i.e., floors/counters/exam tables may still be wet).

In the United States, non-healing wounds affect 3-6 million patients and account for more than 25 billion dollars spend on treatment each year. Wound and surgical dressings are often used to treat, cover and protect wounds and surgical incisions. Wound and surgical dressings come in various forms. For example, for simple cuts, adhesive bandages are typically used. Cotton gauze is also commonly used to cover wounds and surgical incisions. For more serious wounds and surgical incisions, the wound or surgical dressing may include multiple layers of fibrous material with a fluid impervious layer or backsheet to prevent exudates from seeping through the dressing. Typically, medicaments are often manually applied to the wound or surgical dressing before positioning on a wound or surgical incision. A medicament is a medicinal substance or agent. The medicament may include, for instance, an antimicrobial agent or antibiotic agent to encourage healing. Antiseptics are also commonly applied to prevent infection.

There is a need for a less cumbersome and cost-effective method for cleaning and disinfecting biological materials or other contaminants from hard surfaces. Additionally, there is a need for a wound dressing system that stabilizes the wound and prevents deterioration of the wound. Such a system can provide a barrier to the environment, can remove or prevent the growth of microorganisms, such as bacteria, and can provide barriers and or absorbency to combat bodily fluid loss, among other desired outcomes of its use.

### SUMMARY OF THE DISCLOSURE

In various embodiments, an absorbent binder composition can have a superabsorbent polymer material comprising at least 15 percent by mass monoethylenically unsaturated carboxylic acid, sulphonic acid, or phosphoric acid, or salts thereof and an acrylate or methacrylate ester that contains an alkoxysilane functionality; and from about 0.02 to about 0.3 percent by mass of a polyvalent metal cation having a valance of at least two.

In various embodiments, the polyvalent metal cation comprises calcium, copper, zinc, manganese, cobalt, or magnesium. In various embodiments, the polyvalent metal cation comprises calcium chloride, copper (II) chloride, zinc chloride, manganese (II) chloride, cobalt (II) chloride or magnesium sulfate.

In various embodiments, the alkoxysilane functionality forms a silanol functional group which condenses to form a crosslinked polymer on loss of water by evaporation. In various embodiments, the monoethylenically unsaturated carboxylic acid, sulphonic acid, or phosphoric acid, or salts thereof comprises a polyacrylic acid. In various embodiments, the acrylate or methacrylate ester comprises a monomer containing a trialkoxysilane functional group. In various embodiments, the monomer containing a trialkoxysilane functional group comprises at least one of methacryloxypropyl trimethoxy silane, methacryloxyethyl trimethoxy silane, methacryloxypropyl triethoxy silane, methacryloxypropyl tripropoxy silane, acryloxypropylmethyl dimethoxy silane, 3-acryloxypropyl trimethoxy silane, 3-methacryloxypropylmethyl diethoxy silane, 3-methacryloxypropylmethyl dimethoxy silane, 3-(tri-methoxysilyl) propyl methacrylate, or 3-methacryloxypropyl tris(methoxyethoxy)silane.

In various embodiments, the absorbent binder composition further has an antimicrobial agent suitable for use in disinfecting a hard surface. In various embodiments, a method of disinfecting a hard surface can have the steps of spraying the absorbent binder composition onto the hard surface. In various embodiments, the method can further have the step of allowing the absorbent binder composition to dry into a film coating wherein the film coating incorporates any solid or liquid present on the hard surface. In various embodiments, the method can further have the step of removing the film coating from the hard surface. In various embodiments, the method can further have the step retaining the film coating on the hard surface as a protective coating for the hard surface. In various embodiments, the solid or liquid can be selected from at least one of vomit, urine, feces, and blood. In various embodiments, the solid can be a sharp object.

In various embodiments, the absorbent binder composition further has an active agent suitable for use in treating a wound. In various embodiments, the absorbent binder composition can be applied to the wound as a spray. In various embodiments, the absorbent binder composition when on the wound absorbs exudate, blood and debris while rendering disinfection. In various embodiments, the absorbent binder composition can be removed from the wound without damaging the wound bed by either peeling away or by irrigating the wound with copious amounts of saline.

### DETAILED DESCRIPTION OF THE DISCLOSURE

The present invention is directed to an absorbent binder composition that includes a hydrophilic polymer which has the capability of post-application, moisture-induced cross-linking. The absorbent binder composition can include 1) a superabsorbent polymer material which can include at least 15 mass percent monoethylenically unsaturated polymer, such as carboxylic acid, sulphonic acid, or phosphoric acid, or salts thereof, and an acrylate or methacrylate ester that contains an alkoxysilane functionality and 2) a polyvalent metal cation having a valence of at least two. Upon loss of water by evaporation, the alkoxysilane functionality forms a silanol functional group which condenses to form a crosslinked polymer. Upon exposure of the absorbent binder composition to a biological material, such as urine, blood, or feces, the biological material can interact with the polyvalent metal cation and can serve as a catalyst to accelerate the polymer cross-linking and gelation of the polymer.

In various embodiments, the absorbent binder composition can be sprayed directly onto a substrate, a hard surface or a part of a human body and, upon drying, the absorbent binder composition can absorb a contaminant or a biological material. Once the absorbent binder composition has absorbed the contaminant or biological material it can be removed. For example, the absorbent binder composition can be sprayed directly onto a hard surface for removing solid and liquid matter from the hard surface and/or disinfecting the same hard surface. As an additional example, the absorbent binder composition can be sprayed directly onto a wound to absorb and remove exudate from the wound. As another example, the absorbent binder composition can be sprayed onto a substrate which can be used as a wipe to clean a solid or liquid matter from a surface.

The absorbent binder composition can be made by polymerizing monoethylenically unsaturated monomers, one or more of which contains an alkoxysilane functionality. The polymerization can be induced by a variety of initiation techniques including thermal initiation, radiation initiation, or redox chemical reactions. Various types of effective radiation include ultraviolet, microwave, and electron-beam radiation. The initiator generates free radicals to cause polymerization of the monomers. The resultant copolymer includes latent moisture-induced crosslinking capability by incorporation of the alkoxysilane functionality. Moisture induced cross-linking may be accomplished through hydrolysis of the alkoxysilane and subsequent condensation. Incorporation of the polyvalent metal cation having of a valence of at least two into the absorbent binder composition and exposure of the absorbent binder composition to a biological material can accelerate the speed of the cross-linking and gelation of the polymer. The absorbent binder composition can be applied in a flowable state to a substrate or other end use application.

A method for forming such an absorbent binder composition can include providing an absorbent binder composition which, in various embodiments, can have a viscosity that permits delivery of the absorbent binder composition through a commonly available low cost conventional hand sprayer or spray bottle. In various embodiments, the viscosity of the absorbent binder composition can be less than about 10,000 cP and greater than about 500 cP. In various embodiments, the viscosity of the absorbent binder composition can be from about 500 or 650 cP to about 1,000, 2,000, or 10,000 cP. The viscosity of the absorbent binder composition is measured at 16 hours according to the test procedure outlined in U.S. Pat. No. 7,312,286. As explained therein, the viscosity of the absorbent binder composition is measured using a Brookfield DVII+ Programmable viscometer which is available from Brookfield Engineering, Middleboro, Mass., USA. About 200-250 ml of the absorbent binder composition is taken in a 25-ounce plastic cup. The viscometer is generally zeroed initially with a desired Spindle. For the absorbent binder composition, Spindle Number 3 is used. The viscosity is measured at 20 RPM and at temperature of 22±1 degrees C.

A superabsorbent polymer material suitable for use in the absorbent binder composition of the present disclosure can be described as a superabsorbent binder polymer solution such as described in U.S. Pat. No. 6,849,685 to Soerens et al., U.S. Pat. No. 7,312,286 to Lang et al., and U.S. Pat. No. 7,335,713 to Lang et al., the entirety of each of these references is herein incorporated by reference. The superabsorbent polymer material described therein is capable of post-application, moisture-induced crosslinking. Whereas most superabsorbent polymers require the addition of an internal crosslinker to reinforce the polymer, the absorbent binder composition of the present disclosure does not require the addition of a crosslinking agent because the organic monomers act as an internal crosslinker. The internal crosslinker allows the absorbent binder composition to be formed by coating the water-soluble polymer onto the desired surface and then removing the water to activate the latent crosslinker. Additionally, Soerens et al., in U.S. Pat. No. 6,737,491, the entirety of which is herein incorporated by reference, describes an absorbent binder composition of a monoethylenically unsaturated polymer and an acrylate or methacrylate ester that contains alkoxysilane functionality. Also described in Soerens et al. is a method of making the absorbent binder composition that includes the steps of preparing a monomer solution, adding the monomer solution to an initiator system, and activating a polymerization initiator within the initiator system.

The composition disclosed in the references noted above is the reaction product of at least 15 percent by mass monoethylenically unsaturated carboxylic acid, sulphonic acid, or phosphoric acid, or salts thereof, and an acrylate or methacrylate ester that contains an alkoxysilane functionality which, upon loss of water by evaporation, forms a silanol functional group which condenses to form a crosslinked polymer.

In various embodiments, the monoethylenically unsaturated monomer can be acrylic acid. Other suitable monomers include carboxyl group-containing monomers: for example, monoethylenically unsaturated mono- or poly-carboxylic acids, such as (meth)acrylic acid (meaning acrylic acid or methacrylic acid; similar notations are used hereinafter), maleic acid, fumaric acid, crotonic acid, sorbic acid, itaconic acid, and cinnamic acid; carboxylic acid anhydride group-containing monomers: for example, monoethylenically unsaturated polycarboxylic acid anhydrides (such as maleic anhydride); carboxylic acid salt-containing monomers: for example, water-soluble salts (alkali metal salts, ammonium salts, amine salts, etc.) of monoethylenically unsaturated mono- or poly-carboxylic acids (such as sodium (meth)acrylate, trimethylamine (meth)acrylate, triethanolamine (meth)acrylate), sodium maleate, methylamine maleate; sulfonic acid group-containing monomers: for example, aliphatic or aromatic vinyl sulfonic acids (such as vinylsulfonic acid, allyl sulfonic acid, vinyltoluenesulfonic acid, styrene sulfonic acid), (meth)acrylic sulfonic acids [such as sulfopropyl (meth)acrylate, 2-hydroxy-3-(meth)acryloxy propyl sulfonic acid]; sulfonic acid salt group-containing monomers: for example, alkali metal salts, ammonium salts, amine salts of sulfonic acid group containing monomers as mentioned above; and/or amide group-containing monomers: for example, vinylformamide, (meth)acrylamide, N-alkyl (meth)acrylamides (such as N-methylacrylamide, N-hexylacrylamide), N,N-dialkyl (meth)acrylamides such as N,N-dimethylacrylamide, N,N-di-n-propylacrylamide), N-hydroxyalkyl (meth)acrylamides [such as N-methylol (meth)acrylamide, N-hydroxyethyl (meth)acrylamide], N—N-dihydroxyalkyl (meth)acrylamides [such as N,N-dihydroxyethyl (meth)acrylamide], vinyl lactams (such as N-vinylpyrrolidone).

Suitably, the amount of monoethylenically unsaturated carboxylic acid, sulphonic acid, or phosphoric acid, or salts thereof, relative to the weight of the absorbent binder composition may range from about 15 to about 99.9 weight percent. In various embodiments, the monoethylenically unsaturated carboxylic acid, sulphonic acid, or phosphoric acid, or salts thereof, levels may be from about 15, 25, 30 or 50% to about 70, 80, 90 or 99.9% of the weight of the absorbent binder composition. The acid groups can be neutralized to the extent of at least about 25 mol percent, that is, the acid groups can be present as sodium, potassium, or ammonium salts. In various embodiments, the degree of neutralization can be at least about 50 mol percent.

Organic monomers capable of co-polymerization with monoethylenically unsaturated carboxylic acid, sulphonic acid, or phosphoric acid, or salts thereof, which monomers contain a trialkoxysilane functional group or a moiety that reacts with water to form a silanol group, are useful in practice of this invention. The trialkoxysilane functional group has the following structure:

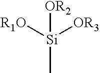

wherein R₁, R₂ and R₃ are alkyl groups independently having from 1 to 6 carbon atoms. The term “monomer(s)” as used herein includes monomers, oligomers, polymers, mixtures of monomers, oligomers and/or polymers, and any other reactive chemical species which is capable of co-polymerization with monoethylenically unsaturated carboxylic acid, sulphonic acid, or phosphoric acid, or salts thereof. In various embodiments, ethylenically unsaturated monomers containing a trialkoxysilane functional group are appropriate and can include acrylates and methacrylates. A particularly desirable ethylenically unsaturated monomer containing a trialkoxysilane functional group is methacryloxypropyl trimethoxysilane, commercially available from Dow Corning, Midland, Mich., under the trade designation Z-6030 Silane. Other suitable ethylenically unsaturated monomers containing a trialkoxy silane functional group include, but are not limited to, methacryloxyethyl trimethoxy silane, methacryloxypropyl triethocy silane, methacryloxypropyl tripropoxy silane, acryloxypropylmethyl dimethoxy silane, 3-acryloxypropyl trimethoxysilane, 3-methacryloxypropylmethyl diethoxy silane, 3-methacryloxypropylmethyl dimethoxy silane, and 3-methacryloxypropyl tris(methoxyethoxy)silane. However, it is contemplated that a wide range of vinyl and acrylic monomers having trialkoxy silane functional groups or a moiety that reacts easily with water to form a silanol group, such as chlorosilane or an acetoxysilane, provide the desired effects are effective monomers for copolymerization in accordance with the present invention.

In addition to monomers capable of copolymerization that contain a trialkoxysilane functional group, it is also feasible to use a monomer capable of copolymerization that can subsequently be reacted with a compound containing a trialkoxysilane functional group or a moiety that reacts with water to form a silanol group. Such a monomer may contain, but is not limited to, an amine or an alcohol. An amine group incorporated into the copolymer may subsequently be reacted with, for example, but not limited to, (3-chloropropyl)trimethoxysilane. An alcohol group incorporated into the copolymer may subsequently be reacted with, for example, but not limited to, tetramethoxysilane.

The amount of organic monomer having trialkoxysilane functional groups or silanol-forming functional groups relative to the weight of the absorbent binder composition may range from about 0.1 to about 15 weight percent. In various embodiments, the amount of monomer can exceed 0.1 weight percent in order to provide sufficient crosslinking upon loss of water by evaporation. Typically, the monomer addition levels are from about 0.1, 1.0, or 1.5% to about 5.5, 10, or 20% of the weight of the absorbent binder composition.

In various embodiments, the absorbent binder composition may include a copolymerizable hydrophilic glycol containing an ester monomer, for example a long chain, hydrophilic monoethylenically unsaturated esters, such as poly(ethylene glycol) methacrylate having from 1 to 13 ethylene glycol units. The hydrophilic monoethylenically unsaturated esters have the following structure:

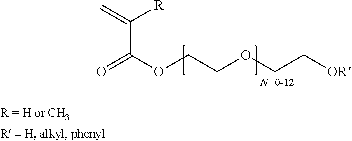

The amount of monoethylenically unsaturated hydrophilic esters relative to the weight of the absorbent binder composition thereof may range from about 0 to about 75 weight percent of monomer to the weight of the absorbent binder composition. In various embodiments, the monomer addition levels are from about 10, 20, or 30% to about 40, 50, or 60% of the weight of the absorbent binder composition.

One of the issues in preparing water-soluble polymers is the amount of the residual monoethylenically unsaturated monomer content remaining in the polymer. For applications in personal hygiene it is required that the amount of residual monoethylenically unsaturated monomer content of the absorbent binder composition be less than about 1000, 500 or 100 ppm. U.S. Pat. No. 7,312,286 discloses at least one method by which an absorbent binder composition may be manufactured so that the residual monoethylenically unsaturated monomer content is at least less than 1000 parts per million. The analysis of residual monoethylenically unsaturated monomer is determined according to the Residual Monoethylenically Unsaturated Monomer Test which is disclosed in U.S. Pat. No. 7,312,286. More specifically, the residual monoethylenically unsaturated monomer analysis is carried out using solid film obtained from the absorbent binder composition. By way of example for this test description, the monoethylenically unsaturated monomer is acrylic acid. High performance liquid chromatography (HPLC) with a SPD-IOAvp Shimadzu UV detector (available from Shimadzu Scientific Instruments, U.S.A) is used to determine the residual acrylic acid monomer content. To determine the residual acrylic acid monomer content, about 05 grams of cured film is stirred in 100 ml of a 0.9% NaCl solution for 16 hours using a 3.5 cm long×0.5 cm wide magnetic stirrer bar at 500 rpm speed. The mixture is filtered and the filtrate is then passed through a Nucleosil C8 100A reverse phase column (available from Column Engineering Inc., U.S.A.) to separate the acrylic acid monomer. The acrylic acid monomer elutes at a certain time with a detection limit at about 10 ppm. The peak area of resulting elutes calculated from the chromatogram is then used to calculate the amount of residual acrylic acid monomer in the film. Initially, a calibration curve can be generated by plotting the response area of pure acrylic acid elutes against its known amount (ppm). A linear curve with a correlation coefficient of greater than 0.996 is obtained.

The absorbent binder composition can further include a polyvalent metal cation having a valence of at least two. In various embodiments, the polyvalent metal cation can have a valence of at least three. Examples of polyvalent metal cations having a valence of at least two or three include calcium, copper, zinc, manganese, cobalt and magnesium. Further examples of polyvalent metal cations suitable for use in the absorbent binder composition of the present disclosure include calcium chloride, copper (II) chloride, zinc chloride, manganese (II) chloride, cobalt (II) chloride, and magesium sulfate. In various embodiments, the absorbent binder composition can have from about 0.02, 0.05 or 0.10 to about 0.15, 0.2, 0.25 or 0.3 percent by mass of the polyvalent metal cation. In various embodiments, the absorbent binder composition can have from about 0.1, 0.3, 0.5, 0.7, 1, 1.3, 1.5, 1.7, 2, 2.3 or 2.5 milimole to about 3, 3.3, 3.5, 3.7, 4, 4.3, 4.5, 4.7 or 5 milimole of polyvalent metal cation. In various embodiments, about 1 milimole of polyvalent metal cation can be added to the absorbent binder composition. In various embodiments, an absorbent binder composition including about 1 milimole of polyvalent metal cation can be stable and flowable after at least 1 year of aging at ambient temperature.

The absorbent binder composition may be prepared by adding a solution of the above monomers to an initiator solution, at a suitable temperature to generate free radicals, for example between about 50 and about 90 degrees Celsius. An initiator solution may be prepared by dissolving an initiator in a solvent. Possible solvents include, but are not limited to, alcohols such as ethanol. A variety of initiators may be useful in the practice of this invention. The polymerization initiator may be activated using a variety of methods including, but not limited to, thermal energy, ultraviolet light, redox chemical reactions. A suitable class of initiators are organic peroxides and azo compounds, with benzoyl peroxide and azobisisobutyonitrile (AIBN) as examples.

Compounds containing an O—O, S—S, or N═N bond may be used as thermal initiators. Compounds containing O—O bonds; i.e., peroxides, are commonly used as initiators for polymerization. Such commonly used peroxide initiators include: alkyl, dialkyl, diaryl and arylalkyl peroxides such as cumyl peroxide, t-butyl peroxide, di-t-butyl peroxide, dicumyl peroxide, cumyl butyl peroxide, 1,1-di-t-butyl peroxy-3,5,5-trimethylcyclohexane, 2,5-dimethyl-2,5-bis(t-butylperoxy)hexyne-3 and bis(a-t-butyl peroxyisopropylbenzene); acyl peroxides such as acetyl peroxides and benzoyl peroxides; hydroperoxides such as cumyl hydroperoxide, t-butyl hydroperoxide, p-methane hydroperoxide, pinane hydroperoxide and cumene hydroperoxide; peresters or peroxyesters such as t-butyl peroxypivalate, t-butyl peroctoate, t-butyl perbenzoate, 2,5-dimethylhexyl-2,5-di(perbenzoate) and t-butyl di(perphthalate); alkylsulfonyl peroxides; dialkyl peroxymonocarbonates; dialkyl peroxydicarbonates; diperoxyketals; ketone peroxides such as cyclohexanone peroxide and methyl ethyl ketone peroxide. Additionally, azo compounds such as 2,2′-azobisisobutyronitrile abbreviated as AIBN, 2,2′-azobis(2,4-dimethylpentanenitrile) and 1,1′-azobis(cyclohexanecarbonitrile) may be used as the initiator.

An aqueous solution of the polyvalent metal cation can be incorporated into the absorbent binder composition. In various embodiments, the absorbent binder composition can have from about 0.02, 0.05 or 0.10 to about 0.15, 0.2, 0.25 or 0.3 percent by mass of the polyvalent metal cation. In various embodiments, the absorbent binder composition can have from about 0.1, 0.3, 0.5, 0.7, 1, 1.3, 1.5, 1.7, 2, 2.3 or 2.5 milimole to about 3, 3.3, 3.5, 3.7, 4, 4.3, 4.5, 4.7 or 5 milimole of polyvalent metal cation. In various embodiments, about 1 milimole of polyvalent metal cation can be added to the absorbent binder composition. In various embodiments, the polyvalent metal cation can be added to the liquid absorbent binder composition with stirring at ambient temperature. In various embodiments, the polyvalent metal cation can be dissolved in water first to create a solution which can then be added to the liquid absorbent binder composition.

In various embodiments, the absorbent binder composition can be poured or sprayed onto the desired surface upon which a biological material (e.g., blood, urine and/or fecal matter) is located. In some instances, the absorbent binder composition can be applied directly onto the area where the absorbent properties are needed. In various embodiments, the absorbent binder composition can be applied to a substrate which can be paper, film, woven materials, nonwoven materials, and combinations thereof. For example, the absorbent binder composition can be applied to a nonwoven material to increase its absorbency and thereby enable the composite material to be used as a wiper for any number of surfaces including, but not limited to, skin. “Nonwoven” refers to materials and webs having a structure of individual fibers or filaments which are interlaid, but not in an identifiable manner as in a knitted fabric. Nonwoven materials and webs have been formed from many processes such as, for example, meltblowing processes, spunbonding processes, air laying processes, and bonded carded web processes. In various embodiments, the absorbent binder composition can be used to treat a hard surface and/or disinfect the same hard surface. In such embodiments, the absorbent binder composition can be applied directly to the hard surface or may be applied to a substrate to wipe the hard surface. In various embodiments, the absorbent binder composition can be used in the treatment of a wound or a different area of a human where absorbency is needed. In such embodiments, the absorbent binder composition can be applied directly to the skin or can be applied to a substrate to wipe the skin. Once the absorbent binder composition is applied to the desired surface upon which a biological material is located, crosslinking can be moisture-induced by hydrolysis and condensation and the biological material can interact with the polyvalent metal cation present in the absorbent binder composition to accelerate the rate of the cross-linking and gelation.

In various embodiments, the absorbent capacity of the absorbent binder composition can be at least 1 gram of fluid per gram of superabsorbent polymer material. In various embodiments, the absorbent capacity of the absorbent binder composition can be at least 3 grams of fluid per gram of superabsorbent polymer material. In various embodiments, the retention capacity of the absorbent binder composition can be greater than 10 or 12 g/g. The absorbent capacity and the retention capacity can be measured using the Centrifuge Retention Capacity Test described in U.S. Pat. No. 7,312,286.

In addition, in various embodiments, modifying agents such as compatible polymers, plasticizers, colorants, and preservatives may be incorporated into the absorbent binder composition. In various embodiments in which a plasticizer is present, the plasticizer may be a hydrophilic plasticizer and may include, but is not limited to, a polyhydroxy organic compound such as glycerin, and low molecular weight polyolefinic glycols such as polyethylene glycol (PEG) of molecular weight ranges from about 200 to about 10,000. The amount of plasticizer relative to the weight of the absorbent binder composition may range from about 0 or 10% to about 40, 60 or 75% by weight of the plasticizer to the weight of the absorbent binder composition. In various embodiments in which a colorant is present, the colorant can provide a visual observation as to whether the absorbent binder composition has been adequately applied and adequately covers the desired target surface.

In various embodiments, the absorbent binder composition described herein can be used in the cleaning and/or disinfecting biological materials from a hard surface. In such embodiments, the absorbent binder composition described herein can further include an antimicrobial agent suitable for use in the cleaning and/or disinfecting of a hard surface.

Suitable antimicrobial agents include quaternary ammonium compounds (didecyl dimethyl ammonium chloride, benzethonium chloride, centrimonium chloride, cetylpyridinium chloride, cocamidopropyl PG-dimonium chloride phosphate, cetrimide, didecyl dimethyl ammonium carbonate, didecyl dimethyl ammonium bicarbonate), peroxides (hydrogen peroxide, urea hydrogen peroxide, benzoyl peroxide, calcium peroxide, magnesium peroxide, zinc peroxide, polyvinylpyrollidone-hydrogen peroxide), surfactants, silver and/or copper particles or ions, biguanides (chlorhexidine digluconate, chlorhexidine diacetate, chlorhexidine dihydrochloride, polyhexamethylene biguanide), isothiazolinones (methylisothiazolinone, methylchloroisothiazolinone, benzisothiazolinone, octylisothiazolinone), alcohols (ethanol, isopropanol), acids (benzoic acid, boric acid, citric acid, lactic acid, malic acid, maleic acid), hypochlorites (sodium hypochlorite, calcium hypochlorite), iodine, phenolics (chloroxylenol, hexachlorophene, triclosan, salicylic acid, thymol, o-phenylphenol, cresols), potassium monopersulfate, chlorine dioxide, anilides (triclocarban, tribromsalan), pyrithiones, and antimicrobial peptides. For instance quaternary ammonium compounds (otherwise referred to as “quats”) include benzalkonium chloride (USP Mason Chemical, U.S.A.). In various embodiments, suitable peroxides include organic peroxides such as hydrogen peroxide (Sigma-Aldrich Chemical Co., U.S.A.). In various embodiments, suitable silver materials include silver nitrate, silver oxide, and silver metal particles (e.g., SILVAGARD®, available from Halyard Health, U.S.A). In various embodiments, suitable copper materials include copper nitrate, copper chloride, and copper sulfate.

Ingredients capable of manipulating the release kinetics of the antimicrobial agent may also be present, including but not limited to polymers and salts. Polymer and salt selection is dependent upon which antimicrobial agent(s) is present in the absorbent binder composition.

In various embodiments, the absorbent binder composition described herein can be used in the treatment of a wound in skin, such as human skin. In such embodiments, the absorbent binder described herein can further include an active agent.

In various embodiments, the active agent can include gases, antimicrobial agents, including but not limited to, anti-fungal agents, anti-bacterial agents, anti-viral agents, and anti-parasitic agents, mycoplasma treatments, growth factors, proteins, nucleic acids, angiogenic factors, anaesthetics, mucopolysaccharides, metals and other wound healing agents.

Active agents can include, but are not limited to, gases, such as nitrogen, carbon dioxide, and noble gases, pharmaceuticals, chemotherapeutic agents, herbicides, growth inhibitors, anti-fungal agents, anti-bacterial agents, antiviral agents, and anti-parasitic agents, mycoplasma treatments, growth factors, proteins, nucleic acids, angiogenic factors, anaesthetics, mucopolysaccharides, metals, wound healing agents, growth promoters, indicators of change in the environment, enzymes, nutrients, vitamins, minerals, carbohydrates, fats, fatty acids, nucleosides, nucleotides, amino acids, sera antibodies and fragments thereof, lectins, immune stimulants, immune suppressors, coagulation factors, neurochemicals, cellular receptors, antigens, adjuvants, radioactive materials, and other agents that affect cells or cellular processes.

Examples of anti-microbial active agents can include, but are not limited to, isoniazid, ethanbutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, and silver salts such as chloride, bromide, iodide, and periodate.

Growth factor agents can include, but are not limited to, basic fibroblast growth factor (bFGF), acidic fibroblast growth factor (aFGF), nerve growth factor (NGF), epidermal growth factor (EGF), insulin-like growth factors 1 and 2 (IGF-1 and IGF-2), platelet derived growth factor (PDGF), tumor angiogenesis factor (TAF), vascular endothelial growth factor (VEGF), corticotropin releasing factor (CRF), transforming growth factors α and β (TGF-α and TGF-β), interleukin-8 (IL-8), granulocyte-macrophage colony stimulating factor (GM-CSF), the interleukins and the interferons.

Acid mucopolysaccharides can include, but are not limited to, heparin, heparin sulfate, heparinoids, dermatitin sulfate, pentosan polysulfate, chondroitin sulfate, hyaluronic acid, cellulose, agarose, chitin, dextran, carrageenan, linoleic acid, and allantoin.

Proteins, which can be useful in the treatment of compromised tissues, such as wounds, can include, but are not limited to, collagen, cross-linked collagen, fibronectin, laminin, elastin, and cross-linked elastin or combinations and fragments thereof. Adjuvants, or compositions that boost an immune response may also be used in conjunction with the wound dressings.

Other wound healing agents may include, but are not limited to, metals such as zinc and silver.

A number of absorbent binder composition samples were formed and tested with the test results reported in Table 1. The superabsorbent polymer material used in each of the samples was obtained from Evonik Stockhausen, LLC (Greensboro, N.C., U.S.A.) and is an oligomeric polyacrylic acid containing internal silanol cross-linkers and is a flexible absorbent binder, with the designation FAB, and which is manufactured in accordance with U.S. Pat. No. 7,312,286. FAB is an aqueous solution of sodium polyacrylate. For each of the samples, a polyvalent metal cation was dissolved in 1 ml of deionized water and then added to 5 grams of this superabsorbent polymer material and stirred for three minutes. The amount of each polyvalent metal cation added is listed in Table 1. To test the impact of blood on the samples of absorbent binder compositions, each sample of absorbent binder composition was placed on a petri dish and 2 drops of blood were added to the indicated absorbent binder composition samples with the results reported in Table 1.

TABLE 1

| | | | | Visual Observation |
|---|---|---|---|---|
| | | | | Following Addition of 2 |
| | | Amount of Metal | Visual Observation | drops Blood to 1 ml of |
| Sample | Polyvalent | Cation added to | of FAB/Metal Cation | FAB/Metal Cation |
| No. | Metal Cation | 5 g FAB | Composition | Composition |
| 1 | None | 0 g | Clear Liquid | No gel formation observed |
| | | | | after 8 hours |
| 2 | Calcium | 1 mmole, 0.11 g | Hazy Liquid | Gels on contact |
| | Chloride | | | |
| 3 | Copper (II) | 1 mmole, 0.17 g | 10% gel/90% liquid | Gels on contact |
| | Chloride | | | |
| 4 | Iron (II) | 1 mmole, 0.20 g | Gels on contact | Sample was already a gel |
| | Chloride | | | |
| 5 | Zinc Chloride | 1 mmole, 0.134 g | Clear liquid | Gels on contact |
| 6 | Manganese (II) | 1 mmole, 0.126 g | Clear liquid | Gels on contact |
| | Chloride | | | |
| 7 | Cobalt (II) | 1 mmole, 0.13 g | Clear ruby colored | Gels on contact |
| | Chloride | | liquid | |
| 8 | Iron (III) | 1 mmole, 0.27 g | Gels on contact | Sample was already a gel |
| | Chloride | | | |
| 9 | Magnesium | 1 mmole, 0.24 g | Clear liquid | Gels on contact |
| | Chloride | | | |

The absorbent binder compositions containing the calcium chloride, copper (II) chloride, zinc chloride, manganese (II) chloride, cobalt (II) chloride, and magnesium chloride each demonstrated the ability to experience an accelerated cross-linking and gelation when exposed to the blood. The absorbent binder composition that did not contain the polyvalent metal cation did not gel on contact when exposed to the blood and the absorbent binder compositions containing iron (II) chloride and iron (III) chloride each gelled upon the addition of the iron (II) chloride or the iron (III) chloride to the FAB.

The absorbent binder composition sample numbers 1 and 2 described above were also tested for their reaction to human urine. To each of the samples, 1 ml of human urine was added. The visual observation of Sample 1 was that no gel formation was observed after 8 hours. The visual observation of Sample 2 was that a gel formed on contact with the urine.

The presence of a polyvalent metal cation, particularly any of calcium, copper, zinc, manganese, cobalt, and magnesium, in an absorbent binder composition can result in an increased cross-linking of the absorbent binder composition when exposed to a biological material which can result in faster absorbency of the biological material from the surface upon which the biological material is located.

The following example illustrates the potential application as a spray for wounds.

### Example 1

The leg section of a male mannequin was placed on a laboratory bench and a mixture of calf blood and saline were placed on the mannequin leg to simulate a leg wound. An absorbent binder composition was formulated contain the FAB superabsorbent polymer material, calcium chloride as the polyvalent metal cation and Silvagard® as an antimicrobial agent. The absorbent binder composition contained 50 ml FAB, 1.1 g (10 mmole) calcium chloride, and 10 g (1% solids) of Silvagard® M98 (available from Halyard Health, Alpharetta, Ga., U.S.A.). The absorbent binder composition was sprayed (Prevail sprayer, Chicago Aerosol, Coal City Ill.) over the section of the mannequin leg where the calf blood and saline combination were located. The absorbent binder composition formed a film coating on the section of the mannequin leg where it was sprayed. The film coating was found to set-up quickly providing a good transparent film that sealed the wound. The film coating was also observed to rapidly absorb the calf blood and saline combination. Later, the film coating could be easily removed by either peeling it from the surface of the mannequin leg or by irrigating it with a gentle stream of copious amounts of saline. The irrigation of the film coating caused the film coating to swell and wash away.

The following examples illustrate potential applications for hard surface cleaning and disinfection.

### Example 2

To model clean-up of vomit and/or fecal solids on a hard surface, a 25 g sample of fruit cocktail (Dole) was scattered on a glass surface to cover approximately 12 cm diameter area and simulate a contaminated area. An absorbent binder composition was formulated to contain the FAB superabsorbent polymer material, calcium chloride as the polyvalent metal cation, and Silvagard® as an antimicrobial agent. The absorbent binder composition contained 50 ml FAB, 1.1 g (10 mmole) calcium chloride, and 10 g (1% solids) of Silvagard® M98 (available from Halyard Health, Alpharetta, Ga., U.S.A.). The absorbent binder composition was then sprayed over the contaminated area to cover and coat the liquid and solids. The absorbent binder composition formed a film coating where it was sprayed. After set-up the film was then easily peeled off and away from the surface to leave a clean and dry surface. The film had incorporated and disinfected both the liquid and solids to leave a clean and dry surface.

### Example 3

To model clean-up of hard solids and sharp material on a hard surface, 25 grams of broken quartz glass pieces were scattered onto a hard surface. An absorbent binder composition was formulated to contain the FAB superabsorbent polymer material, calcium chloride as the polyvalent metal cation, and Silvagard® as an antimicrobial agent. The absorbent binder composition contained 50 ml FAB, 1.1 g (10 mmole) calcium chloride, and 10 g (1% solids) of Silvagard® M98 (available from Halyard Health, Alpharetta, Ga., U.S.A.). The absorbent binder composition was sprayed liberally over the area having the broken glass fragments and allowed to set-up into a film coating. The solid film coating that was formed was then easily removed in one piece to leave a clean and dry surface with no sign of any fragments or splinters of glass. The spray coating and resultant film coating had incorporated all glass fragments of all sizes. It was also observed that the larger glass fragments were incorporated into the film coating and any projections of sharp edges had been coated by the spray coating and was rendered non-sharp by the film coating. This allows safe removal by the users hand without the risk of being cut.

### Example 4

To model cleaning of the surface and keys of an instrument a Texas Instruments calculator (Model TI-36X Solar, Texas Instruments, Houston Tex.) was used. An absorbent binder composition was formulated to contain the FAB superabsorbent polymer material, calcium chloride as the polyvalent metal cation, and Silvagard® as an antimicrobial agent. The absorbent binder composition contained 50 ml FAB, 1.1 g (10 mmole) calcium chloride, and 10 g (1% solids) of Silvagard® M98 (available from Halyard Health, Alpharetta, Ga., U.S.A.). The absorbent binder composition was sprayed over the top of the calculator including the keys and screen and allowed to set-up into a film coating. After the film coating set-up the film coating was pulled off easily and cleanly to leave a clean and dry surface. It was observed that the film coating had covered and gone around the keys as the removed film coating had imprints where the keys had been covered. This showed that the film coating had fully covered all surfaces to remove dust and/or food particles and also disinfected the surfaces.

### Example 5

The following example shows the release of cleaning and/or disinfecting agents from an absorbent binder composition. A variety of absorbent binder compositions were formulated to contain the FAB superabsorbent polymer material, calcium chloride, and an antimicrobial agent. The absorbent binder composition contained 50 ml FAB and 1.1 g (10 mmole) calcium chloride, and the antimicrobial agent as noted in Table 2 below. The absorbent binder compositions were then analyzed according to the Kirby-Bauer Antibiotic test method which is a Zone of Inhibition test method (This test method is also known by the American Association of Textile Chemists and Colorists (AATCC) as test method 147-1998.). In this example, the antimicrobial agents incorporated into an absorbent binder composition were Benzalkonium chloride, hydrogen peroxide, and a combination of the Benzalkonium chloride and hydrogen peroxide. According to the test method, the absorbent binder compositions were then brought into contact with test wafers to impregnate the test wafers with the absorbent binder compositions. The absorbent binder composition-impregnated test wafers were then brought into contact with various pathogenic bacteria on an agar plate and the agar plates were then left to incubate according to the test method. Following incubation, the distance between the wafers and the growth of bacteria was measured. Table 2 shows the results of the testing and illustrates that the incorporation of an antimicrobial agent into an absorbent binder composition provides an effective release and disinfection capability to the film coating formed by the absorbent binder composition.

TABLE 2

| | P. aeroginosa | E. coli | S. aureus |
|---|---|---|---|
| | Zone of | Zone of | Zone of |
| | Inhibition (cm) | Inhibition (cm) | Inhibition (cm) |
| 5% Benzalkonium | 0.0 | 3.0 | 4.4 |
| chloride in FAB | | | |
| 1.25% Benzalkonium | 0.0 | 3.5 | 4.7 |
| chloride in FAB | | | |
| 3% hydrogen peroxide | 0.0 | 3.9 | 5.3 |
| in FAB | | | |
| 5% Benzalkonium | 3.9 | 5.9 | 7.0 |
| chloride with 3% | | | |
| hydrogen peroxide in | | | |
| FAB | | | |
| Negative Control | | 0.0 | |
| (untreated test wafer) | | | |
| Positive Control | | 1.9 | |
| (n = 1) | | | |
| (vancomycin wafer) | | | |
| Desiccation Control | | 0.0 | |
| (n = 2) (dry wafer) | | | |

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

What is claimed is:

1. An absorbent binder composition characterized by comprising:
a. a superabsorbent polymer material comprising:
i. at least 15 percent by mass monoethylenically unsaturated carboxylic acid, sulphonic acid, or phosphoric acid, or salts thereof;
ii. an acrylate or methacrylate ester that contains an alkoxysilane functionality;
b. from about 0.02 to about 0.3 percent by mass of a polyvalent metal cation having a valance of at least two.

2. The absorbent binder composition of claim 1 wherein the polyvalent metal cation comprises calcium, copper, zinc, manganese, cobalt, or magnesium.

3. The absorbent binder composition of claim 1 wherein the polyvalent metal cation comprises calcium chloride, copper (II) chloride, zinc chloride, manganese (II) chloride, cobalt (II) chloride or magnesium sulfate.

4. The absorbent binder composition of claim 1, wherein the alkoxysilane functionality forms a silanol functional group which condenses to form a crosslinked polymer on loss of water by evaporation.

5. The absorbent binder composition of claim 1 wherein the monoethylenically unsaturated carboxylic acid, sulphonic acid, or phosphoric acid, or salts thereof comprises a polyacrylic acid.

6. The absorbent binder composition of claim 1 wherein the acrylate or methacrylate ester comprises a monomer containing a trialkoxysilane functional group.

7. The absorbent binder composition of claim 6 wherein the monomer containing a trialkoxysilane functional group comprises at least one of methacryloxypropyl trimethoxy silane, methacryloxyethyl trimethoxy silane, methacryloxypropyl triethoxy silane, methacryloxypropyl tripropoxy silane, acryloxypropylmethyl dimethoxy silane, 3-acryloxypropyl trimethoxy silane, 3-methacryloxypropylmethyl diethoxy silane, 3-methacryloxypropylmethyl dimethoxy silane, 3-(tri-methoxysilyl) propyl methacrylate, or 3-methacryloxypropyl tris(methoxyethoxy)silane.

8. The absorbent binder composition of claim 1 further comprising an antimicrobial agent suitable for use in disinfecting a hard surface.

9. A method of disinfecting a hard surface, the method comprising the step of spraying the absorbent binder composition of claim 8 onto the hard surface.

10. The method of claim 9, further comprising the step of allowing the absorbent binder composition to dry into a film coating wherein the film coating incorporates any solid or liquid present on the hard surface.

11. The method of claim 10 further comprising the step of removing the film coating from the hard surface.

12. The method of claim 10 further comprising the step of retaining the film coating on the hard surface as a protective coating for the hard surface.

13. The method of claim 10 wherein the solid or liquid can be selected from at least one of vomit, urine, feces, and blood.

14. The method of claim 10 wherein the solid can be a sharp object.

15. The absorbent binder composition of claim 1 further comprising an active agent suitable for use in treating a wound.

16. The absorbent binder composition of claim 15, wherein said absorbent binder composition can be applied to the wound as a spray.

17. The absorbent binder composition of claim 15, wherein said absorbent binder composition when on the wound absorbs exudate, blood and debris while rendering disinfection.

18. The absorbent binder composition of claim 16, wherein said absorbent binder composition can be removed from the wound without damaging the wound bed by either peeling away or by irrigating the wound with copious amounts of saline.
